(19) United States
(12) Patent Application Publication
(10) Pub. No.: US 2017/0014372 A1
(43) Pub. Date: Jan. 19, 2017

(54) Drug Combination for Treatment of Proliferative Diseases

(71) Applicant: ECOLE POLYTECHNIQUE FEDERALE DE LAUSANNE (EPFL), Lausanne, OT (CH)

(72) Inventors: Patrycja Nowak-Sliwinska, Sergy (FR); Paul Dyson, Ecublens (CH); Arjan Griffioen, Amsterdam (NL); Andrea Weiss, Lausanne (CH); Hubert Van den Bergh, Goumoens-la-Ville (CH); Xianting Ding, Shanghai (CN)

(21) Appl. No.: 15/124,039

(22) PCT Filed: Mar. 12, 2015
(86) PCT No.: PCT/EP2015/055246

§ 371 Date: Sep. 7, 2016

(30) Foreign Application Priority Data
Mar. 13, 2014 (IB) PCT/IB2014/059747

(51) Int. Cl. A61K 31/28; A61K 31/4745; A61K 31/517

(52) U.S. Cl. CPC A61K 31/28; A61K 31/517; A61K 31/4745

(57) ABSTRACT

Angiogenesis inhibitory drug combination obtained according to a specific algorithm, preferably a FSC, in which an initial combination of drugs is iteratively adjusted. The drug combination according to the invention may advantageously comprise a RAPTA-C compound. In a more specific case the combination comprises RAPTA-C and eriotinib.

### FIELD OF INVENTION

The present invention relates to the treatment of proliferative diseases, such as cancer, atherosclerosis, arthritis and age-related macular degeneration, and more precisely to the identification of drug combinations that may be used in these fields of medicine.

### STATE OF THE ART

The use of targeted therapies is currently a widely implemented approach for cancer therapy (1-3). Their actual contribution to the prolongation of overall patient survival, however, is still rather limited due to genetic heterogeneity (4) and the development of drug resistance (5, 6). Therefore, noticeably more emphasis is being placed on the development of therapies that aim to inhibit specific cancer-related targets. Angiogenesis inhibition is an anti-cancer approach that has been limited by drug resistance despite its potential for the development of more efficient therapies. The field of angiogenesis research is quickly developing with the emergence of new targeted agents which provide the potential for a large improvement in therapeutic outcome if an optimal combination of multiple targeted agents can be developed (7). Such combination strategies may lead to increased treatment efficacy due to synergistic interactions between drugs, and may also result in a significant reduction of side effects due to the possibility of major dose reductions (8). It is also anticipated that the development of drug resistance is more difficult when multiple distinct pathways are regulated simultaneously, not sequentially (9). Most current combination strategies employed in the clinic are designed to target complementary pathways, however, compounds are selected based on their previous success as single agents and are administered at doses used during single drug therapy (10). The failure of some of these clinically tested combinations may be attributed to a lack of knowledge on optimal dosing and synergies between compounds. A systematic search for an optimal drug combination from a broad array of angiostatic-targeted approaches would be of therapeutic value.

### SUMMARY OF INVENTION

Angiogenesis is intricately regulated through a system of highly robust and redundant cell signaling pathways (11). Targeting multiple different signaling pathways may allow drug combinations to be identified, which synergistically inhibit angiogenesis through the lateral inhibition of multiple targets in non-overlapping pathways (12).

The inventors have screened for optimized combinations of multiple targeted drugs regulating a broad array of cellular functions and intracellular pathways. As individual compounds at various dose-ratios can significantly affect the efficacy of a drug combination, the parametric space in the screening of combinations can be huge (e.g. 9 drugs at 6 concentrations provides 6⁹ combinations).

The present invention provides a rapid identification of the most potent drug-dosage combinations with a minimal amount of experimental effort. This is achieved by the use of a algorithm in which a combination of drugs is iteratively adjusted.

The algorithm is preferably a Feedback System Control (FSC) (FIG. 1A). FSC attempts to optimize a cellular output (i.e. inhibition of angiogenic cell processes) by iteratively adjusting a combination of drugs based on the experimental results. As long as the cellular output does not satisfy the optimization goal, a feedback search algorithm, e.g. the differential evolution algorithm, is used to modify the input drug-dosage combinations for the following set of experiments. The iterative feedback loop continues to optimize the input variables until the optimization goal is reached. Endothelial cells line the interior of blood vessels and are highly active in the process of angiogenesis, which requires their migration and proliferation towards an angiogenic stimulus. For this reason, the pharmacological inhibition of endothelial cell activity was selected as a means to inhibit tumor angiogenesis.

According to a first embodiment of the invention an initial optimization with the FSC technique using a large set of drugs is being performed. A second optimization with a subset of drugs is subsequently performed, after certain compounds were eliminated based on the analysis of drug interactions using response surface modeling with second-order linear regression. Based on this approach, the inventors surprisingly discovered a unique combination of three small molecule-based drugs that selectively inhibit EC function while having minimal effects on tumor or healthy cell function. An in vivo model of human ovarian carcinoma demonstrated that tumor growth inhibition is correlated with a lack of tumor vascularization. This strategy may lead to effective anti-angiogenic cancer treatment via specific targeting of the tumor endothelium.

The invention provides in particular the following opportunities and/or advantages:

The translation of in vitro testing into (pre)clinical treatment.

Improvement of the anti-angiogenic activity by combining different drugs with angiostatic activity.

Decreasing the adverse side effects while only improving on angiostatic activity.

Decreasing of drug-resistance by reducing the dose of the components in a drug mixture, while only improving on angiostatic activity.

### DETAILED DESCRIPTION OF THE INVENTION

The invention will be better understood below, by way of examples. Of course the invention is not limited to those examples.

### EXAMPLE 1

### FSC-Guided Optimization Identified the Most Synergistic Drug Combinations.

The initial drug optimization was performed using the feedback system control (FSC) technique for the inhibition of endothelial cell (EC) proliferation (FIG. 1A). An array of nine drugs (anginex 1, bevacizumab 2, axitinib 3, erlotinib 4, anti-HMGB1 Ab 5, sunitinib 6, anti-vimentin Ab 7, RAPTA-C 8, BEZ-235 9) targeting a broad spectrum of angiogenesis pathways was selected for this study. Broad single drug dose-response curves were prepared based on endothelial cell proliferation and migration assays (FIG. 8A). Dose-response curves were used to identify three coded doses for each compound: dose 3, representing the dose where 10% inhibition based on the control is observed (ED₁₀), dose 2, representing the dose where 5% inhibition is observed (ED₅), and dose 1, representing half the maximal dose where no effect is observed (ED₀) and are provided in Table 1. Using the above-mentioned dose levels, the multi-drug optimization was performed using the FSC technique for EC proliferation inhibition testing 19 drug combinations per iteration (FIG. 1A). FSC allowed for the progressive improvement of the average output, which decreases after each iteration (FIG. 1B). The optimization was terminated after iteration 10, as a plateau was reached with no improvement in the best identified combination for three iterations. All results were used in the second-order linear regression model in order to eliminate compounds for the subsequent search based on the assessment of individual drug contributions and interactions. This model generated single drug linear and quadratic effects, as well as two-drug interactions for all two-drug pairs (FIG. 8B, R²=0.75). Based on the coefficient analysis of the regression model, the following compounds excluded from further study: 1 and 6 due to poor single drug linear and quadratic effects, as well as mostly antagonistic drug interactions with other compounds), and 2 (due to a lack of inhibitory single drug linear and quadratic effects). Although compound 6 interacts synergistically with 7 and 9, these interactions were minimal compared to antagonistic interactions. Finally, compound 5 was excluded due strong antagonistic interactions with compounds 8 and 9, and compound 7 was eliminated due to a stimulatory single drug quadratic effect and antagonism with compound 9.

The regression coefficients for the stepwise linear regression model are provided in FIG. 1C. This model confirms the selection of compounds 3 (axitinib), 4 (erlotinib), 8 (RAPTA-C) and 9 (BEZ-235). Drug interaction terms provided minimal contributions to the overall inhibition of ECRF24 proliferative activity.

In an independent optimization, the inventors investigated the activity of the same drugs for the inhibition of EC migration. Drug doses were adapted based on the dose-response curves of the single drugs in the migration assay (FIG. 9A). As a result of eleven iterations performed in this optimization, the best output value obtained corresponds to approximately 40% activity of migration (FIG. 9B), however, it did not indicate synergistic drug interactions based on the calculation of the combination index (CI) lower than 0.8 (14). The data from this optimization was modeled using a second order linear regression model (FIG. 9C). Comparing the regression coefficients, 4 and 9 had the strongest overall contributions, and would be well paired with compounds 1 and 8 or 6 and 7.

### Refined Four-Drug Search Leads to Optimal Synergistic Drug Combination.

In the next step the FSC was used to test the interaction between the four selected compounds, i.e. 3, 4, 8 and 9. The 50 best performing drug combinations on EC inhibition of proliferation are shown in FIG. 2A (for less effective drug combinations, see FIG. 10A). The most effective drug combinations, which indicated synergistic drug interactions were composed of either: 4, 8 and 9 (combinations A, B, D, E, F) only 4 and 8 (H), at varying dose ratios. Combinations C and G contained compound 3 that had CI>0.8.

The obtained data was modeled using a second-order linear regression model (R²=0.73) (FIG. 3B). The single drug linear contribution of all compounds was significant (solid arrows), whereas only compound 4 (erlotinib) showed a significant inhibitory single drug quadratic effect. Although the only significant two-drug interaction effect was seen between 3 and 4 (indicated by *, FIG. 3B), 3 was also contributed a stimulatory (positive) second-order single drug contribution (arrow with dotted line, FIG. 3B).

### Selected Optimized Drug Mixtures are Endothelial Cell Specific and Apoptosis Inductive

To establish whether the identified drug mixtures have a specific effect on ECs combinations A-H, selected based on the results in FIG. 2A, were further investigated for their activity on primary endothelial (human umbilical cord endothelial cells, HUVEC) and fibroblast (HDFa) cells, freshly isolated PBMCs and five different human tumor cell lines (A2780, 786-0, LS174T, MDA-MB-231 and HT-29) in cell viability assays (FIG. 3).

Single drug cell viability assays were performed on all cell types (FIG. 11). ECRF24 cell viability was not strongly affected by any of the drug concentrations with a maximal single drug efficacy of approximately 25% inhibition of ECRF24 viability. Similar cell viability was seen for single drugs on other primary cells (HDFa and PBMC), with the exception of HUVEC cells, which were more sensitive to 4 and 9. Interestingly, a strong effect for the selected combinations was only observed in the EC cells (ECRF24 and HUVEC) (FIG. 3A), which showed similar levels of inhibition (albeit with stronger single drug effects), while in HDFa and PBMC the maximum inhibitory effect of the most potent drug combinations was around 50% compared to a much greater efficacy of 90% in ECRF24 and HUVEC cells.

Most of the cancer cell lines were less sensitive to the single drugs than the ECRF24 and HUVEC (FIG. 3A), with the exception of 3 and 4 at high dose. Compound 3 at high dose had a relatively strong inhibitory effect on 786-0 cells (approx. 30% inhibition), while at high dose 4 had a relatively potent effect on the A2780 cell line (approximately 40% inhibition). The strongest response of the selected combinations in tumor cells was observed for A2780 cells, which were strongly inhibited by all combinations, except F and H. This may be partially attributed to the increased activity of 4 seen in this cell line when administered at high dose. The 786-0 cell line was also strongly inhibited by some mixtures, particularly C, also possibly related to the increased activity of 3 in these cells. HT29, LS174T and MDA-MB-231 cell lines were not strongly affected by any of the combinations, showing a maximum inhibition of only 50%. Of particular interest is F, which showed a striking difference in activity between the ECRF24 cells and all tumor cell lines, and showed the most potent synergistic interaction (CI=0.4) while using the lowest drug doses.

The inhibitory effect of these drug combinations on cell migration was subsequently tested on ECRF24 and 786-0 cell lines (FIG. 3B). ECRF24 migration was maximally inhibited by F but only by 30%. This confirms the lack of synergistic inhibition of ECRF24 migration that is shown in FIG. 9. The migration inhibition of 786-0 cells was more pronounced (approx. 45% for F) than for ECRF24 cells.

Flow cytometry indicated significantly increased apoptosis induction in ECRF24 cells for combinations B (*p=0.05), C (*p=2E-5), E (*p=0.05), F (*p=0.04) and G (*p=3E-5) relative compared to the control (FIG. 3C). The highest apoptosis level was obtained for C and was approximately two-fold lower than apoptosis induced by sunitinib (10 μM, used here as a positive control).

ECRF24 were harvested and protein lysates were subjected to Western blot analysis including downstream effectors such as panAKT, pMAPK and ribosomal protein S6 (pS6) (FIG. 4). For combinations A, B, and F, the expression of all three effectors was inhibited as compared to sham treated (CTRL) cells. The expression pS6 appears to be inhibited by all of the compounds, particularly BEZ235, and is very low in combination containing BEZ235 as well. Of interest is the very minimal difference in protein expression between combinations A and B, where the dose of RAPTA-C is twice lower. In case of H, pS6 panAKT expression is reduced as compared to the control.

In vivo tumor growth inhibition indicated anti-angiogenic and anti-tumoral mechanisms of optimized drug combination.

The inventors further tested drug combinations in vivo on a human ovarian carcinoma (A2780) grafted on the chorioallantoic membrane (CAM) of the chicken embryo and human colorectal adenocarcinomas (LS174T) grafted intradermally in Swiss nu/nu mice (FIG. 5). A27980 tumors were treated with combinations A, B, F, G and H which were adapted to this model while maintaining the drug dose ratios identified in vitro (i.e. limiting the maximal activity of any single compound's activity; in this case compound 9 was the limiting compound, with an activity of approximately 35% at its applied concentration) and combinations J, K, and L randomly composed from the four drugs (Table 2). Treatment was performed by intravenous injection of the drug combinations at the specified doses on treatment days 1 and 2. Combinations A, B, F, G and H were selected as they have the most promising in vitro effects and the strongest indications of synergy. Additionally, these combinations had varying effects on the viability of A2780 cells, allowing us to identify the combinations that act specifically on the inhibition of angiogenesis or potentially through both anti-angiogenic and anti-tumor mechanisms. Applied on their own, compounds 4 and 8 inhibited tumor growth by approximately 6% (at low concentration of 2.9 and 307 μg/kg, respectively) and by 20% (at high concentration 29 and 615 μg/kg, respectively). For these combinations, a dose corresponding to approximately 35% tumor growth inhibition was used for compound 9. The treatment with combinations A, B, F and H resulted in 58%, 48%, 68%, and 53% tumor growth inhibition, respectively, compared to control tumors (**p<0.01, N 5, FIG. 5A). Additional randomly select mixtures, labeled J-L, were also tested. Of these combinations, the four-drug combinations J wasvery effective resulting in synergistic (*p=0.03; CI=0.7) tumor growth inhibition by 87% (J). Highly effective three-drug combinations were also identified, such as mixture L, which inhibited tumor growth by 82% (**p=0.03; CI=0.2). Combination L contained compounds 4+8+9, while a less effective three-drug combination (K) containing compound 3+4+9 inhibited tumor growth only 50% (CI=1). Of the mixtures tested, the strongest synergistic effect was seen in combinations F, G, and L, (CI=0.45, 0.46, 0.18; respectively), all of which contained compounds 4+8+9 (F and L) or only 8+9 (G).

It was observed that with altering dose ratio between 4, 8 and 9 in corresponding drug combinations (A, B, F, L) the effective tumor growth inhibition could be gained (L).

On the last day of the experiment A2780 tumors (FIG. 5B) were resected, and paraffin embedded for immunohistochemical staining for endothelial cells (CD31) (FIG. 5C). While control tumors were well vascularized, microvessel density was significantly lower in tumors treated with F (*p=0.04), FIG. 5C. Interestingly, even though A contained higher doses of compounds than B, F or H, its administration resulted in the least effective inhibition of tumor growth and anti-angiogenic activity.

Based on these results and modeling results from the 4-drug optimization in vitro, the inventors used human colorectal adenocarcinoma (LS174T) grafted intradermally in Swiss nu/nu mice) to study further the interaction of 4+8+9. Mice were randomized into groups and treated with sham (CTRL), individual drugs, 4+8+9 (M1 and M2), and 4+8 (M3), respectively (Table 2) Individual drugs inhibited tumor growth only by approximately 0% and 6% (4) at 5 and 15 mg/kg, 10% (8) or 23% (9), whereas combinations M1 and M2 by 76±14% and 25±17%, respectively (FIG. 5D). On the last day of the experiment (FIG. 5E) tumors were resected, and paraffin embedded for immunohistochemical staining for endothelial cells (CD31). In M1-treated tumors microvessel density was approximately 80% lower then in sham-treated tumors (CTRL), FIG. 5F.

### EXAMPLE 2

Synergistic Activity of Erlotinib in Combination with RAPTA-C in Endothelial Cells.

Synergistic effects of RAPTA-C and erlotinib in endothelial cells (RF24) have been identified. This synergistic activity (combinatory index, CI<1) for multiple RAPTA-C and erlotinib dose combinations was not observed in tumor cells, i.e. human colorectal carcinoma LS174T, human ovarian carcinoma A2780, human renal cell carcinoma 786-0, human lung carcinoma SW173 or A549 (see Table 3).

It has been further observed, for selected erlotinib/RAPTA-C combinations, significant increases in apoptosis induction in RF24 cells (FIG. 6).

In order to determine the anti-tumor activity of erlotinib/RAPTA-C combinations in vivo the preclinical model of human ovarian A2780 carcinoma grown on the chicken chorioallantoic membrane CAM was used (15) (FIG. 7). Embryos were randomized and treated within the following groups via intravenous administration: 0.14% DMSO (CTRL), RAPTA-C (100 μM; 2.2 mg/kg, 20 μl), erlotinib (5 μM; 0.02 mg/kg, 20 μl), or their mixture (1:1; 2.2: 20 μl), each day for two consecutive days corresponding to treatment days 1 and 2. Tumor growth curves are presented in FIG. 2. The combination of erlotinib/RAPTA-C premixed and administrated i.v. at the monotherapy doses, inhibited tumor growth synergistically leading to tumor size reduction by 57% vs. CTRL, as measured at the last day of the experiment (CI=0.71).

These results confirm that the simultaneous co-administration of erlotinib/RAPTA-C induces an anti-tumor effect, acting probably via an anti-angiogenic mechanism.

### Renal Cell Carcinoma Specific Drug Combinations

An optimization screen performed on the inhibition of renal cell carcinoma (Caki-1) cell proliferation initiated with 10 different targeted compounds resulted in the identification of the 8 best drug combinations containing 3 to 4 drugs presented in Table 4.

All combinations contained both erlotinib+RAPTA-C. The addition of AZD4547, which is the fibroblast growth factor receptor inhibitor, resulted in effective and synergistic inhibition of Caki-1 cell proliferation. Results of screening these combinations on 786-0 cells and non-cancerous embryonic kidney cells (HEK239, not shown) did not result in any synergistic activity (Table 4).

### Discussion

In example 1 the inventors used a simple assay for the inhibition of endothelial cell (EC) viability to show that a unique set of high efficacy drug combinations can be identified from a large set of compounds using the FSC technique. In only 10 iterations, the number of compounds being considered in the optimization has been reduced from nine to four, retaining the compounds which had the most profound inhibitory effect on EC viability based on the assessment of drug contributions and interactions through second order linear regression modeling of the obtained data in the iterations. The elimination of certain compounds is of particular interest, as it validates the ability of the data modeling to identify synergistically or antagonistically interacting compounds. Regression models led to interesting observations. Compound 2 (bevacizumab) was included in the screen due to its pivotal clinical role as an angiogenesis inhibitor, even though it was likely to have little to no activity in an in vitro setting (16). This was indeed seen in the regression models, where both the first and second order single-drug effects of 2 are not statistically significant (in Suppl. FIG. 1B) and do not appear in the stepwise linear regression model (FIG. 1C). Also of note is the interaction of 3 and 6. As these compounds have very similar target profiles and are competing for the same drug target (17), they are not likely to interact synergistically, competing for the same target and should likely not be used together in anoptimized combination. Indeed, an antagonistic two-drug interaction has been observed (Suppl. FIGS. 1B) and 6 was excluded from further optimization. The exclusion of 6 over 3 is also logically justifiable, as 3 is a second generation TKI with fewer targets and a relatively stronger affinity for its targets, possibly explaining its stronger first-order linear effect when compared to 6. The analysis revealed significant inhibitory single drug linear contributions from compounds 3, 4, 8 and 9 (FIG. 1B). Additionally, analysis of drug interactions based on the data obtained from the optimization of ECRF24 migration inhibition lead to the elimination of similar compounds. Compounds 4 and 9 appeared to contribute most strongly based on single drug linear and quadratic effects and share mainly synergistic interaction with 6 and 7. Based on this approach, a unique combination of three small molecule-based drugs that selectively inhibit EC function while having minimal effects on tumor or healthy cell function has been identified.

Subsequent optimization of the refined set of four compounds allowed for the identification of highly effective drug combinations (with up to 90% inhibitory activity) using few compounds at relatively low individual doses. The therapeutic potential of such mixtures, which can achieve effective inhibition of cell viability with decreased drug doses can be seen when considering the reduction of individual drug doses, possible when used in the combination. In order to obtain the same level of EC proliferation inhibition as is achieved in the best combination (i.e. 90% inhibition) doses of approximately 50 μM, 2000 μM and 120 nM would be required for compounds 4, 8, 9, respectively, assuming linear dose-response curves. This represents a theoretical dose reduction of 5, 11, and 6-fold for each compound in combination, Evaluation of selected combinations in various cell lines showed the most potent activity of any drug combination in both of the EC types (ECRF24 or HUVEC), while the effects of single drugs were generally not more potent in these cells. This indicates that the optimized drug combinations result in selective inhibition of EC proliferation. Additionally, the potential for drug dose reduction in drug combinations and the relatively limited effects of all single drugs and drug combinations on primary cell types (HDFa and PBMC) indicates the possibility of effective treatment with minimized side-effects using such an optimized, low-dose drug combination.

Based on the in vitro results the drug combinations composed of drugs 3, 4, 8 and 9 were tested in ovarian carcinoma xenografts on the CAM. Analysis of the most synergistic of these tested combinations lead to the subsequent additional elimination of compound 3 and further testing of combinations containing compounds 4, 8 and 9 in a colorectal carcinoma xenograft model in mice. Treatment with these drugs combinations resulted in significant tumor growth inhibition, based on measurement of tumor volume as compared to control (sham treated) tumors (FIG. 5). Based on IHC analysis it has been observed that tumor growth inhibition was linked to significant anti-angiogenic activity.

The identified mixtures indicated synergism between 4 (erlotinib; an EGFR inhibition), 8 (RAPTA-C), and 9 (BEZ235; mTOR inhibitor). Although the mechanism of action of RAPTA-C is not yet fully understood (18, 19) the combination of mTOR and EGFR inhibitors has already been identified as a synergistic combination in various cell cancer types (20, 21) and in the inhibition of tumor growth in vivo (22). It was already shown that while erlotinib may inhibit Akt and S6 in sensitive cell lines, the mTOR inihibitor, rapamycin, could fully inhibit S6 in all cell lines tested, however through the activation of Akt phosphorylation. Furthermore, the combination of erlotinib and rapamycin in certain cell lines allowed for the inhibition of upstream activation of Akt, possibly explain the synergism seen when combining these molecules (23). Results of western blot analysis of ECRF24 cells revealed a slight decrease in pAkt protein in cells exposed to compounds 4 (erlotinib) and 8 (RAPTA-C) and the nearly complete inhibition of pS6 expression by the mTOR inhibitor compound 9 (BEZ235), possibly explaining the synergistic relationship seen between these compounds. Additionally, compound 3 resulted in an increase in pAkt, which may have actually further upregulated the mTOR-induced activation of AKT, explaining the reduced activity seen in combinations containing this compound. Notably, three-drug combinations (such as N) can result in better overall anti-tumor activity than the two-drug combinations (H) and even four-drug combinations (G).

Moreover, in this study the feedback system control technique has proven to robustly identify a potent dug combination, as previously shown on other optimizations (13, 24, 25). Analyzing the data obtained separately in in vitro assays on EC proliferation or migration, suggests that EC proliferation might, depending on the experimental setup, be a limiting process over cell migration in angiogenesis, a conclusion that has been proposed by others (26) based on the computer modeling of angiogenic processes in tumor inhibition.

The optimized drug combination composed of erlotinib, RAPTA-C and BEZ235 appeared to have superior activity in the experimental models. It has been showed that an overall tumor growth inhibition is driven by apoptosis induction in ECRF24 and tumor vasculature growth inhibition. The best optimzed drug combination (M1) let to 80% LS174T tumor growth inibition, wheras the drug combinations composed with the same drugs in vitro (A, B, D, F) only insignificantly inhibited the LS174T cell proliferation (FIG. 3A). On the other hand the same drug combinations tested in A2780 tumors grafted on the CAM inhibited the over all tumor growth with reduced microvessel density, thus confirming their in vitro ativity in ECs. Therefore, together with quantification of IHC CD31 positive stainings, it has been confirmed that the anti-tumor effect of M1 was driven by the anti-angiogenic activity.

It was also very interesting to note that RAPTA-C was previously reported as anti-metastatic (27) and anti-angiogenic (18), but practically not active in treatment primary tumor (27). It has been observed results show that RAPTA-C administrated at low concentrations simultanously with erlotinib and BEZ235 synergistically inhibits tumors growth via anti-angiogenic effect.

Summarizing, the use of FSC enabled fast and reliable search of a potent drug combination without a knowledge of exact effect of individual drug. It has been not only confirmed that the optimized drug combination was composed of much lower doses that individual doses, but also proved that RAPTA-C in combination with other compounds can effectively inhibit human ovarian carcinoma or colorectal adenocarcinoma growth.

In example 2, it has beeb showed that erlotinib+RAPTA-C combinations are endothielial cell specific and induce synergistic endothelial cell viability inhibition. This anti-angiogenic activity leads to the synergisitic A2780 tumor growth inhibition in vivo.

In the screen on renal cell carcinoma Caki-1 cells, expressing wild-type VHL tumor-suppressor protein, optimal three-drug combinations were identified, all containing RAPTA-C and synergistically inhibited Caki-1 cells viability. Simultanously, other human renal cell carcinoma cells 786-0, VHL negative, were not sensitive to these drug combinations.

### BRIEF DESCRIPTION OF THE FIGURES

FIG. 1. Optimization of the inhibition of endothelial cell proliferation.

FIG. 2. Overview of optimization of the 4-drug combination for the inhibition endothelial cell proliferation.

FIG. 3. Selection and validation of the best drug combination.

FIG. 4. Analysis of protein expression by western blot for panAKT, pMAPK and pS6.

FIG. 5. Inhibition of A2780 (A-C) or LS174T (D-F) tumor growth in vivo by the best mixtures optimized.

FIG. 6. Apoptosis induction in RF24 cells incubated with erlotinib (e), RAPTA-C (r), or their combinations.

FIG. 7. In vivo anticancer activity of erlotinib (5 uM) /RAPTA-C (100 uM) combination in human ovarian carcinoma tumors (A2780) grown on the CAM.

FIG. 8. Single-drug assays on EC and migration performed over a large range of concentrations. Data in relation with ECRF24 proliferation inhibition.

FIG. 9. EC migration inhibition optimization.

FIG. 10. Drug combinations tested in EC proliferation assay.

FIG. 11. The activity of individual drugs at the tested concentrations on various healthy and cancerous cell lines.

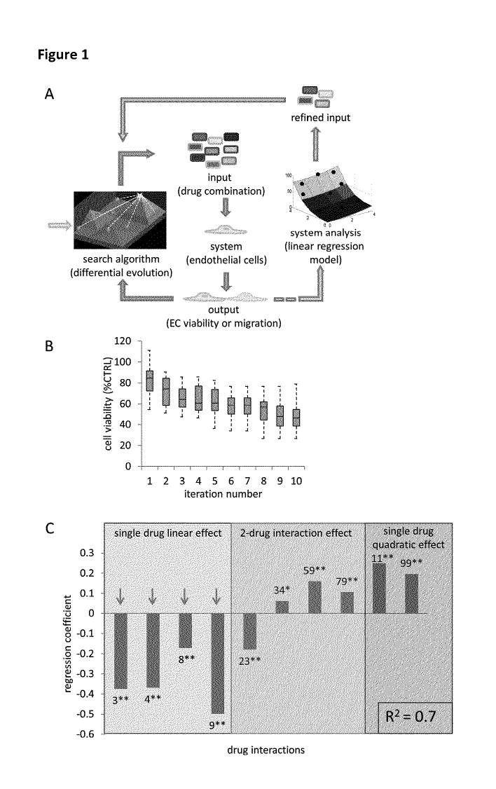

FIG. 1. Optimization of the inhibition of endothelial cell proliferation. (A) An overview of the FSC technique used to optimize the drug combination in vitro. A closed-loop process was used starting with randomly selected combinations of drugs and implementing a search algorithm and in vitro cell assays to find an optimized system output. After achieving a plateau in the system output, the data obtained from the optimization is used to model the system, analyze drug interactions and eliminate certain drugs. Using a refined set of drugs, the drug combination is again optimized in the closed-loop cycle. (B) Box plot providing information on the output (in vitro EC viability inhibition, represented as percent of control) of the 19 most potent drug combinations identified by the end of each iterative cycle of the FSC optimization. The plot shows the progressive reduction in the average output after each iteration. The upper and lower bars represent the mixtures with the highest and lowest outputs. The lowest output corresponds to the best-identified drug combination, which was identified at iteration 8 and did not improve for the last 3 iterations. (C) Regression coefficients obtained from the stepwise linear regression model generated by modeling data obtained from the optimization of ECRF24 proliferation inhibition (R²=0.7). The upper and left-hand side arrows indicate drug contributions, which inhibit EC proliferation. *indicates p-value <0.05 and ** indicates p-value <0.01.

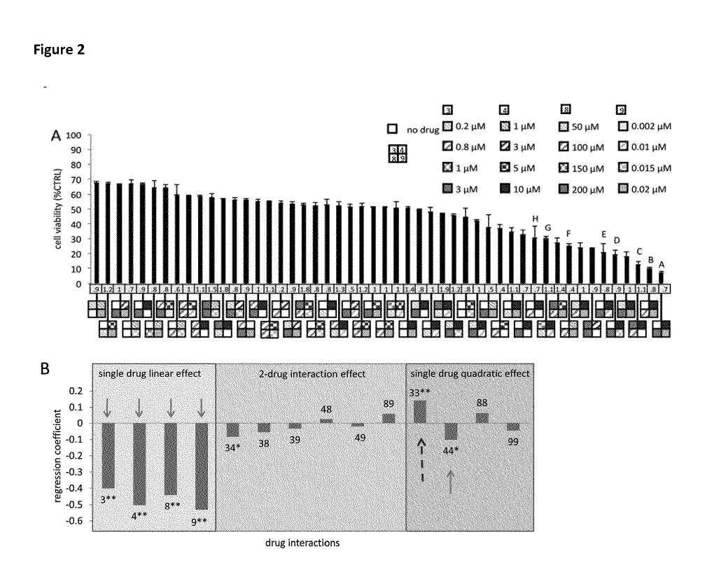

FIG. 2. Overview of optimization of the 4-drug combination for the inhibition endothelial cell proliferation. (A) Four drug combination optimization. Using the concentrations of each small molecule drug corresponding to coded doses of “1” through “4” (defined by the legend at the top right) where combinations were tested in EC proliferation assay. The 50 best performing combinations with their corresponding ‘combination index’ or ‘CI’ values calculated using Compusyn®, which indicates synergistic drug interactions for combinations with a ‘CI’ less than or equal to 0.8. The square icons present the specific combinations, where each position in the square and color corresponds to a specific drug (i.e. position 1 represents axitinib) and the concentrations of each compound are represented by the different patterns. The most promising combinations were selected and labeled A-H. The data represent a mean of at least 2 independent experiments, with 3 replications each, and error bars represent the SEM. (B) The regression coefficients for the quadratic linear regression model generated based on the data from optimization of the refined four-drug combination (R²=0.73). *indicates significance p-value <0.05 and ** indicates significance p-value <0.01.

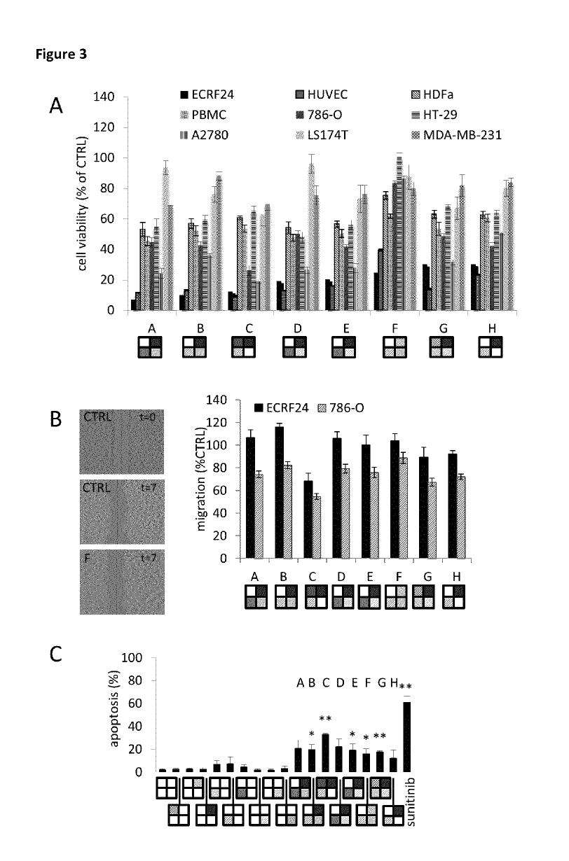

FIG. 3. Selection and validation of the best drug combination. (A) The effects of the most promising 8 drug combinations (A-H from FIG. 2) were tested on the proliferation of various healthy and cancerous cell lines and (B) on the migration of EC-RF24 and 786-0 cells. (C) The effects of individual compounds and combinations on cell apoptosis of ECRF24.

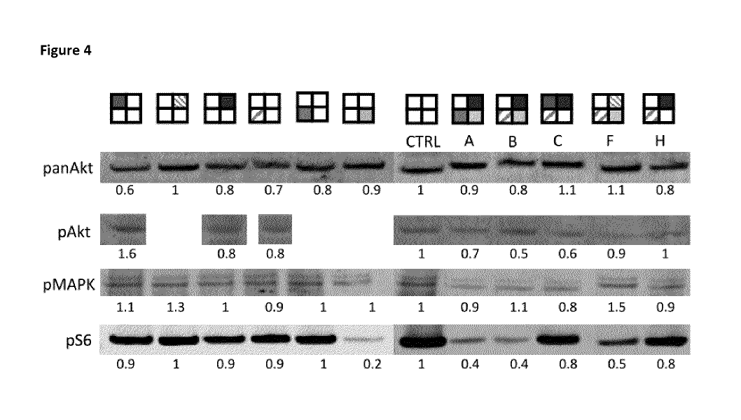

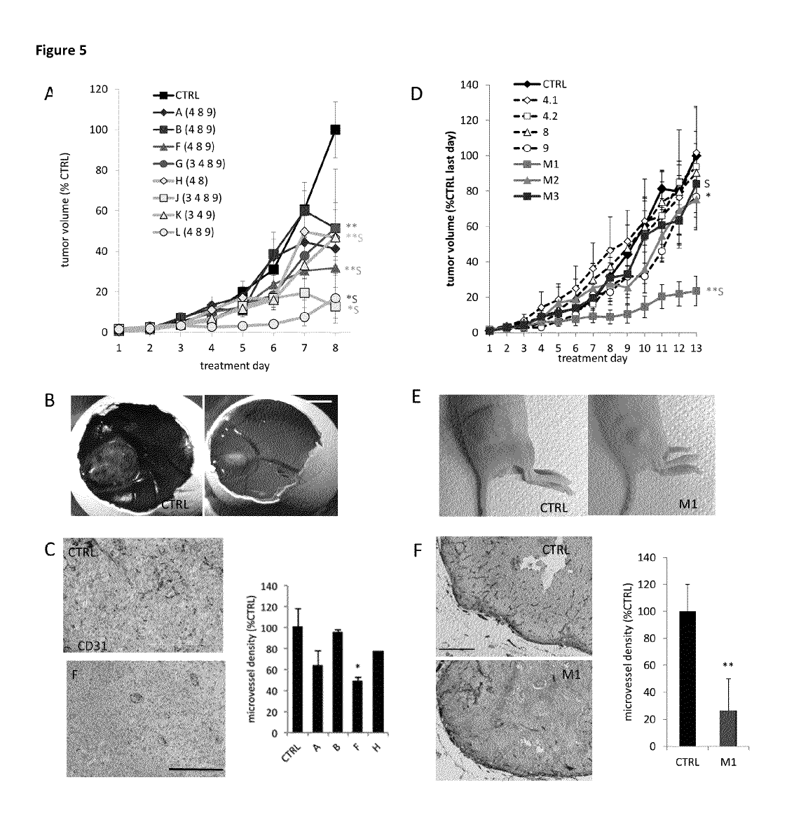

FIG. 4. Analysis of protein expression by western blot for panAKT, pMAPK and pS6. Western blot quantification was performed using dosimetry analysis in ImageJ and represents the mean of at 2 independent experiments. Error bars represent the SEM. *p<0.05 and **p<0.01.

FIG. 5. Inhibition of A2780 (A-C) or LS174T (D-F) tumor growth in vivo by the best mixtures optimized. (A) Growth curve of A2780 tumors grafted on the CAM showing tumor volume with respect to treatment day. Treatment was administered i.v. on days 1 and 2 and tumors were excised on the 8th (last) day of experiment. Data points represent the average tumor volume as a percentage of the final control volume per experiment. (B) Figures show representative tumors of sham treated (CTRL) and F drug combination. (C) The microvessel density profiles are expressed as the number of vessels per mm² and presented as a percentage of a control. (D) LS174T grafted intradermally in Swiss nu/nu mice and treated daily with single compounds or mixtures. The drug combinations composition is listed in Table 2. Data points represent the average tumor volume as a percentage of the final control volume per experiment and error bars represent the SEM; N=3-9. *p <0.05 and **p<0.01. ‘S’ indicates synergy (Cl≦0.8). (E) The microvessel density profiles are expressed as the number of vessels per mm² and presented as a percentage of a control. (F) Immunohistochemical staining of the endothelial cell marker CD31 shows reduced microvessel density in tumors treated with F normalized to the tumor surface area and presented as the percent of control. The bar in the upper right image represents 0.5 mm and is valid for both images. Data points represent average values of 2 independent experiments and error bars represent SEM; N=3-7; **p<0.01.

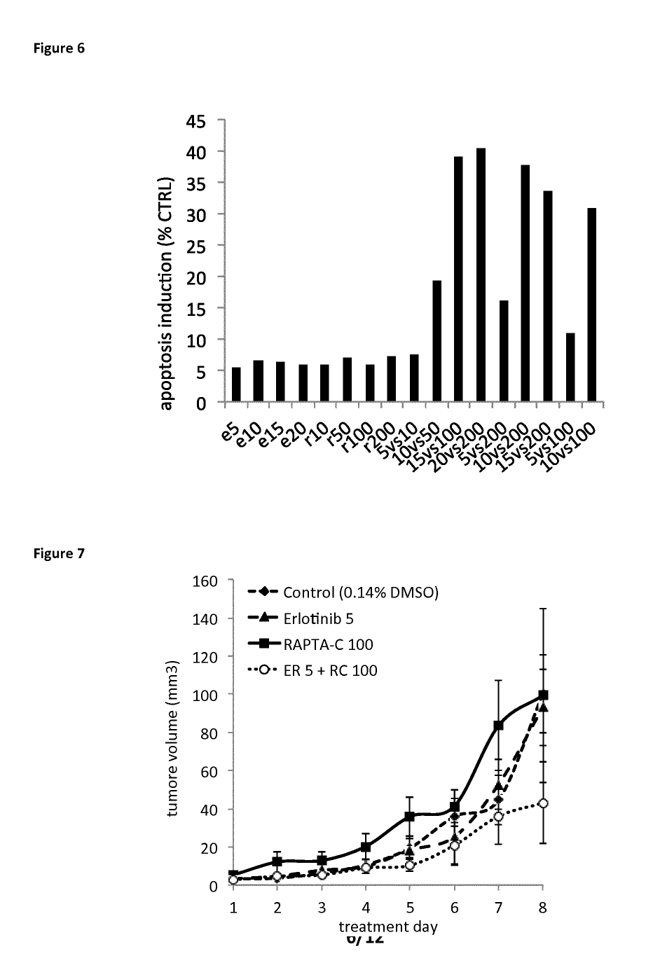

FIG. 6. Apoptosis induction in RF24 cells incubated with erlotinib (e), RAPTA-C (r), or their combinations. Dose ratios are expressed in uM.

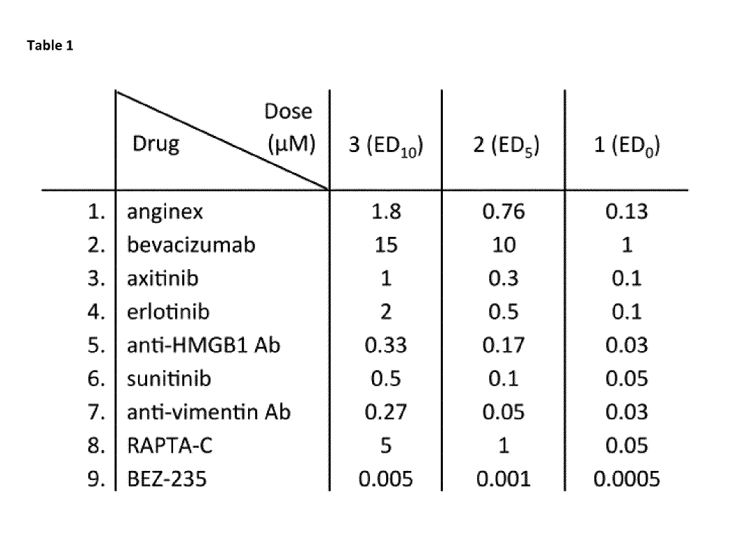

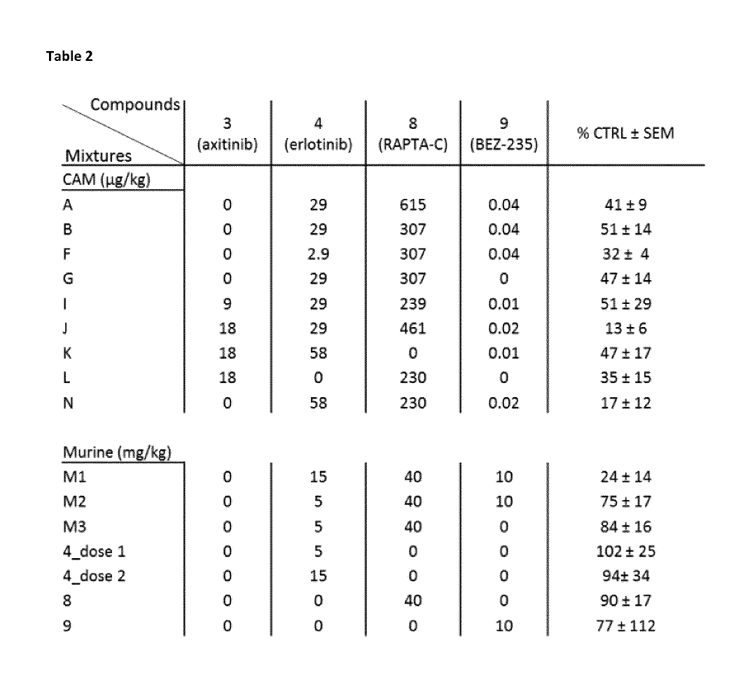

FIG. 7. In vivo anticancer activity of erlotinib (5 uM) /RAPTA-C (100 uM) combination in human ovarian carcinoma tumors (A2780) grown on the CAM. Error bars represent standard error of the mean.

Table 1. Drug dose values used in in vitro assays represented by the coded doses 1, 2 and 3 for each of the nine compounds used in the screen for proliferation inhibition, as well as the reference number used to refer to each compound in the manuscript.

Table 2. Drug dose values used in in vivo assays together with their tumor growth inhibition afficacy. Error bars represent the SEM.

Table 3. Cell line dependent combinatory index for various erlotinib/RAPTA-C combinations.

Table 4. Effective multi-drug combinations (1-8) on inhibition of Caki-1 cells proliferation. Values are given as averages (row “Avg”), with number of examined wells (row “N”) and standard error of the mean (row “SEM”). CI stands for combinatory index. Doses are given in uM.

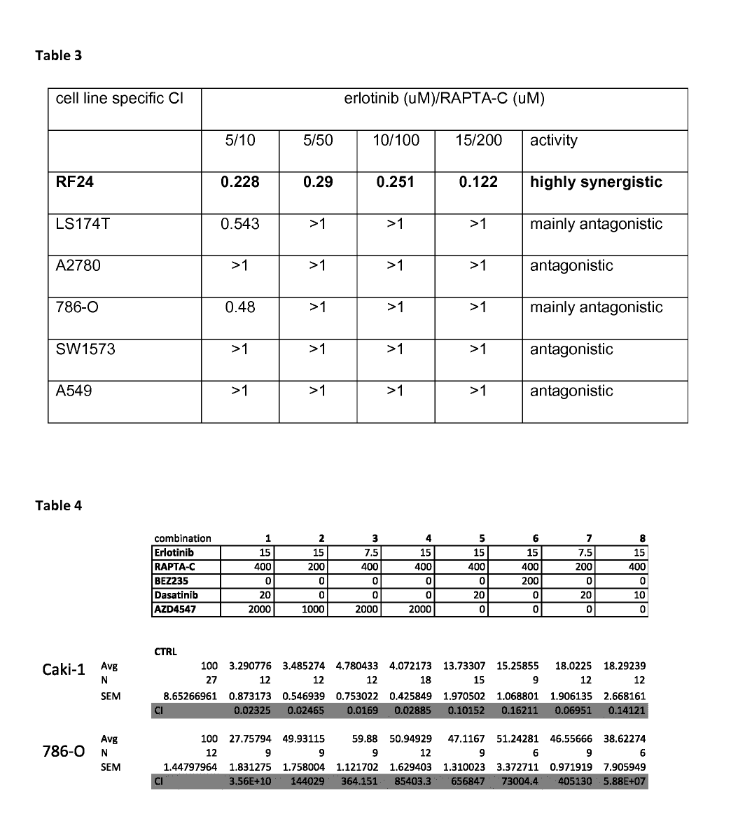

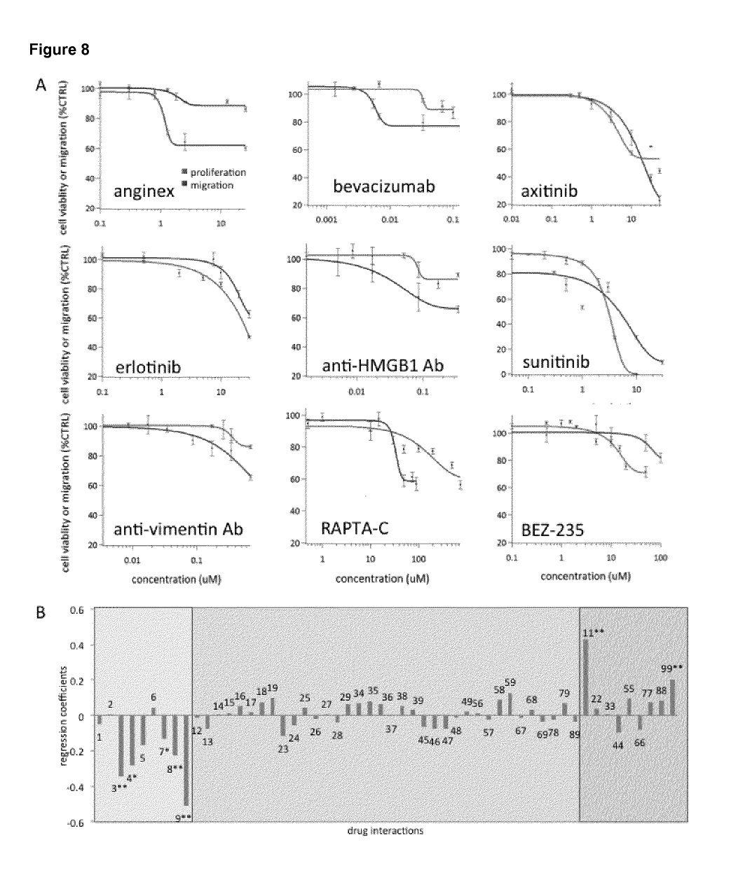

FIG. 8. (A) Results from single-drug assays on EC proliferation and migration performed over a large range of concentrations: 1 (anginex), 2 (bevacizumab), 3 (axitinib), 4 (erlotinib), 5 anti-HMGB1 Ab, 6 (sunitinib), 7 (anti-vimentin Ab), 8 (RAPTA-C) and 9 (BEZ-235). Each data point represents the average of at least two independent experiments, performed in triplicate, and error bars represent the standard error of mean (SEM) of data points. (B) Regression coefficients obtained from the linear regression model generated by modeling data obtained from the optimization of ECRF24 proliferation inhibition.

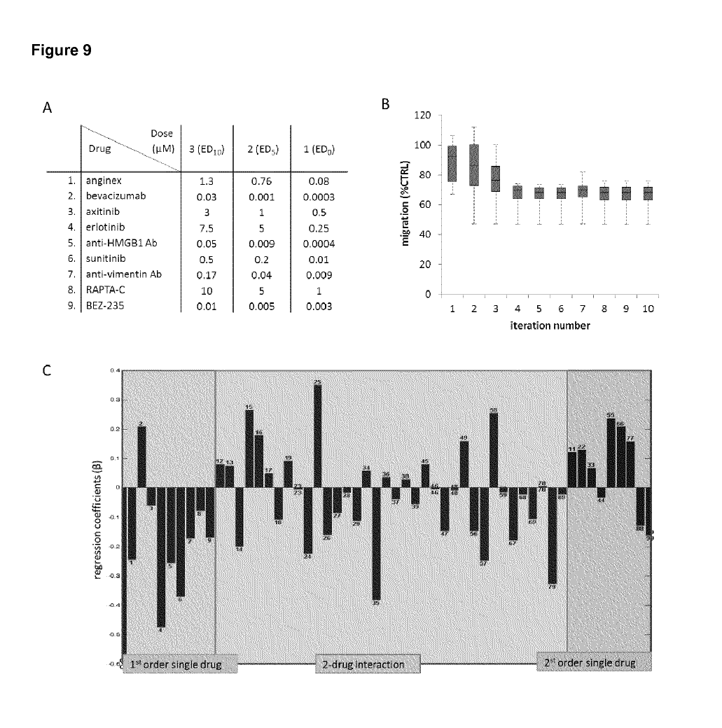

FIG. 9. EC migration inhibition optimization. (A) The real drug doses represented by the coded doses “1”, “2” and “3” for each of the nine compounds used in the combination optimizations for migration inhibition. (B) Each point represents the average output value of the 19 best combinations identified by the end of each iterative cycle. Error bars represent the standard deviation of the same 19 best combinations (not the standard deviation of assays). (C)

The second order two-drug regression coefficients obtained from the quadratic linear regression model

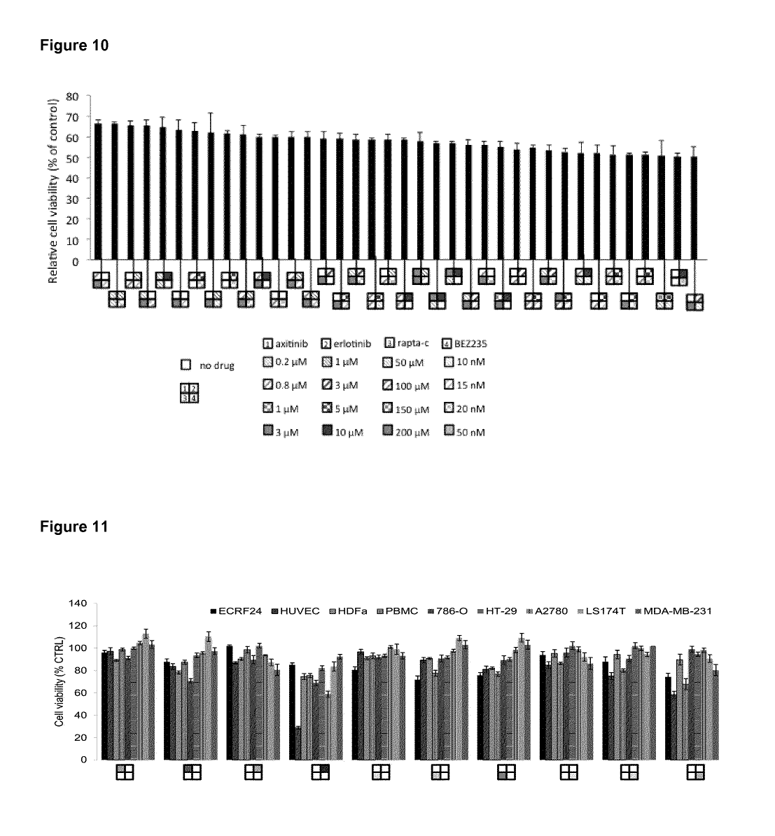

FIG. 10. Drug combinations tested in EC proliferation assay. (A) The graph shows the combinations inhibiting the EC proliferation up to 51% with their corresponding ‘C1’ values calculated using Compusyn®. The square icons present the specific combinations, where each position in the square and color corresponds to a specific drug. The error bars represent the standard deviation.

FIG. 11. The activity of individual drugs at the tested concentrations on various healthy and cancerous cell lines.

### Methods

### Drugs Preparation

Axitinib and erlotinib were purchased from LC laboratories (Woburn, Mass., USA), Sutent® (sunitinib) from Pfizer Inc. (New York, N.Y., USA) and BEZ235 from Chemdea LLC (Ridgewood, USA). RAPTA-C was synthesized and purified as described previously (28). Avastin® (bevacizumab) was obtained from Genentech (San Francisco, Calif., USA). Anti-vimentin monoclonal mouse antibody (clone V9) was purchased from Dako (Glostrup, Denmark) and anti-HMG1 antibody from Santa Cruz Biotechnology (Heidelberg, Germany). Anginex® was provided by Peptx (Excelsior, Mn., USA) and was dissolved in water. The maximum DMSO concentration for any combination maintained at 0.28% (in 0.9% NaCl) and 0.28% DMSO-treated controls were verified as having little to no activity in cell assays.

### Cell Culture and Maintenance

Immortalized human vascular endothelial cells (ECRF24) maintained in medium containing 50% DMEM and 50% RPMI 1640 supplemented with 1% of antibiotics (Life Technologies, Carlsbad, Calif., USA) (Life Technologies, Carlsbad, Calif., USA). ECRF24 were always cultured on 0.2% gelatin coated surfaces. A2780 cells (human ovarian carcinoma) were maintained in RPMI 1640, supplemented as above. Adult human dermal fibroblast (HDFa), 786-0 (renal cell adenocarcinoma), caki-1 (clear cell renal cell carcinoma), HT-29 (colorectal adenocarcinoma), LS174T (colon adenocarcinoma) and MDA-MD-231 (breast adenocarcinoma) cells were maintained in DMEM, supplemented as above. Human umbilical vein ECs (HUVECs) were isolated and cultured as previously described (29). White blood cells (WBCs) were freshly isolated, as previously described (30).

### Cell Viability, Migration, and Apoptosis Assay

Cell viability assay was performed as previously described (31). Cells were seeded in a 96-well culture plate at a density of 2.5-10×10³ cells/well, depending on cell type (ECRF24 in gelatin-coated plates), 24 h prior to the application drug combinations or control conditions (in a total volume of 50 μl) and were subsequently incubated for an additional 72 h. Cell viability was assessed using the CellTiter-Glo luminescent cell viability assay (Promega, Madison, Wisc., USA).

Wound healing assay was performed as previously described (32). EC-RF24 and 786-0 were seeded in 96-well cell culture plates (30×10³ cells/well in 100 μl of cell medium) 24 h prior to making the ‘scratch wounds’ using a sterile scratch tool (Peira Scientific Instruments, Beerse, Belgium) and the application of drug combinations or control conditions. Images were automatically captured on a Leica DM13000 microscope (Leica, Rijswijk, Netherlands) at 5× magnification using Universal Grab 6.3 software (DCILabs, Keerbergen, Belgium). Scratch sizes were determined at T=0 h and T=6 h using Scratch Assay 6.2 (DCILabs), and values reported represented the absolute closure of the scratch would (initial subtracting the final scratch area).

Apoptosis assay was performed as previously described (31). EC-RF24 cells were seeded in a 24-well plate (40×10³ cells/well in 500 μl of cell medium). 24 h later new medium or drug samples were added and cells were grown an additional 72 h. Cells were harvested by trypsinization and incubated with propidium iodide (PI) (20 μg/ml) in buffer containing 2.5 μM citric acid, 45 μM Na₂HPO₄ and 0.1% Triton-X100, pH7.4 for 20 minutes at 37° C. Cells were analyzed with a FACSCalibur (BD Biosciences) in the FL2 channel and apoptotic cells were defined as having subG1 DNA staining.

### The Feedback System Control (FSC) Technique

The feedback system control (FSC) technique was employed as previously described (13, 33, 34). In the research presented here, the FSC technique is implemented using the differential evolution (DE) algorithm (35) and two separate optimization were performed with the cellular outputs of ECRF cell viability (proliferation) and migration (wound healing) assays. 19 drug combinations were tested per iteration and 11 iterations were performed in each optimization, or until a plateau in the best output value was reached.

Drug mixing was performed as follows: stock solutions were first used to prepare the highest concentration of each compound and lower concentrations were prepared through serial dilutions of the higher concentrations. All drug concentrations were prepared 9 times more concentrated than desired to account for dilution by other compounds (or medium when a compound was not included). The drug mixtures were prepared directly before applying drug combinations to cells by first adding the required amount of medium for each combination (i.e.

all compounds which are included at concentration 0) and then adding the required concentration of each compound, always in the same order. The cells were incubated in 50 μl of each mixture for 72 h in the proliferation assay or for 6 h in the migration assay.

### Data Analysis and Modeling

Second order linear regression models were generated using the data obtained from each optimization. Data was modeled using real concentration values and both concentration values and proliferation output data was transformed using the z-score function in Matlab. The negative values of the 2nd-order terms regression coefficients corresponds to synergistic effect, the positive value to the antagonistic effect. Appropriate data and model verification methods were implemented to ensure the accuracy and reliability of predictions made based on these models. The main assumptions of linear regression models were verified (i.e. weak exogeneity, linearity, constant variance, independence of errors, and lack of multicolinearity). As the presence of multicolinearity was indicated in a few of the regressors in the second order linear regression model of the proliferation data (based on the analysis of variance inflation factors (VIF) and condition indices), a stepwise linear regression was also performed to isolate the most important regressors and remove instabilities in the regression analysis due to multicolinearity. Drug interactions were additionally analyzed using the Compusyn software (14), where a ‘combination index’ (CI) is calculated for drug combinations and CI values less than 0.8 indicate synergistic drug combinations, while CI-values greater than 1 indicate antagonistic drug.

In vivo Xenografted Human Ovarian Carcinoma Model on Chicken Chorioallantoic Membrane (CAM)

Human ovarian carcinoma tumors were implanted on the CAM as previously described (36). Briefly, fertilized chicken eggs were incubated in a hatching incubator (37° C. and relative humidity 65%), as previously described (37). On egg development day (EDD) 7, 10⁶ A2780 carcinoma cells were prepared as a spheroid in a 25 μL hanging drop and were transplanted onto the CAM surface 3 h after preparation. Treatment began when vascularized tumors were visible, 3 days after tumor implantation (EDD10). Drug combinations were prepared as done in in vitro assays, were pre-mixed and administered as a 20 μL intravenously injection. Treatment was performed twice (EDD10/11 or treatment days 1/2) and tumor growth was monitored and measured daily, (volume =[larger diameter]x−²×0.52).

### Immunohistochemistry

On the last day of experiment CAM experiments, tumors were resected, fixed overnight in zinc fixative solution as previously described (38). Briefly, 4 μm sections were treated with 0.3% H₂O₂ in methanol for 30 min, followed by a citrate buffer antigen retrieval step (20 min at 95° C.) with blocking by 10% goat serum and 1% BSA. Primary antibody incubation was performed overnight (DIA-310; Dianova, Hamburg, Germany).

### Western Immunoblotting

Cells were seeded in a 6-well plate (30×10⁴ cells per well) 24 h prior to the removal of medium and application of selected drug combinations or DMSO control (total volume of 2 mL). After an additional 5 hrs of incubation at the given conditions, medium was removed, cells were washed with PBS and 100 μl of buffer lysate was added (prepared on ice and composed of RIPA, protease inhibitors (1:1000) and sodium orthovanadate (1:1000)). Wells were then scratched and cell lysate solution was removed and put on ice for 10 min. Cell supernatant was isolated after centrifuging solution at 12,000 min⁻¹ for 15 min. A solution of blue buffer (4×)+u-Page Reducing Agent (10×) was added to supernatant and samples were kept at 95 ° C. for 5 min.

The samples were then run in an electrophoresis gel for 90 min at 125 V. The monoclonal antibodies included phosphorylated Akt (473), total Akt, phosphorylated MAPK, and phosphorylated S6 (235/236 hamster, Cell Signaling). Cell extracts were prepared by detergent lysis [50 mmol/L Tris-HCl (pH 8.0), 150 mmol/L NaCl, 1% NP40, 0.5% sodium deoxycholate, 0.1% SDS containing protease (Sigma, St. Louis, Mo.) and phosphatase (Sigma) inhibitor cocktails]. The soluble protein concentration was determined by micro-BSA assay (Pierce, Rockford Ill.). Protein immunodetection was done by electrophoretic transfer of SDS-PAGE separated proteins to nitrocellulose, incubation with antibody and chemiluminescent second step detection (PicoWest; Pierce). Protein expression was quantified in Fiji by densitometry as previously described (39, 40). Results were expressed as a relative ratio between samples and control.

### Statistical Analysis

Values are given as mean values±standard deviation. Data are represented as averages of independent experiments. Statistical analysis was performed using the student's t-test (in vitro) and Anova (in vivo). *p values lower than 0.05, and **p lower than 0.01 were considered statistically significant.

In addition or instead of RAPTA-C, any other ruthenium-arene based compound of general formula Ru(arene)(X)(Y)(Z)+, where R=any organic group and X, Y and Z=any ligand including chelating ligands and +=0, 1 or 2, can be used in the combination.

Depending on the ruthenium-arene compound used different compound combinations may give different effects in various cancer types and others diseases.

Ruthenium-arene compounds act as a general chemosensitizer that allows them to be used in combination with many different substances. For example, RAPTA-C and other ruthenium-arene compounds can be used in combination with cytotoxic agents such as cisplatin and doxorubicin to treat cancers. Other classes of compounds that can be combined with ruthenium-arene based compound include, but are not limited to, anti-angiogenic, anti-inflammatory, anti-bacterial, anti-viral, anti-fungal compounds.

Ruthenium-arene compounds with drugs may allow known drugs used to treat certain type of disease to be effective in another diseases, e.g. an anti-fungal compound may have anticancer properties when combined with RAPTA-C or other ruthenium-arene compounds.

### REFERENCES

1. Aggarwal S. Targeted cancer therapies. Nature Reviews Drug Discovery. 2010;9:427-8.
2. Chapman P B, Hauschild A, Robert C, Haanen J B, Ascierto P, Larkin J, et al. Improved survival with vemurafenib in melanoma with BRAF V600E mutation. N Engl J Med. 2011 Jun. 30;364(26):2507-16. PubMed PMID: 21639808. Pubmed Central PMCID: 3549296.
3. Gonzalez-Angulo A M, Hortobagyi G N, Ellis L M. Targeted therapies: peaking beneath the surface of recent bevacizumab trials. Nature reviews Clinical oncology. 2011 Jun.;8(6):319-20. PubMed PMID: 21556024.
4. Gerlinger M, Rowan A J, Horswell S, Larkin J, Endesfelder D, Gronroos E, et al. Intratumor heterogeneity and branched evolution revealed by multiregion sequencing. N Engl J Med. 2012 Mar. 8;366(10):883-92. PubMed PMID: 22397650. Epub Mar. 9, 2012 eng.
5. Ebos J M, Lee C R, Kerbel R S. Tumor and host-mediated pathways of resistance and disease progression in response to antiangiogenic therapy. Clin Cancer Res. 2009 Aug 15;15(16):5020-5. PubMed PMID: 19671869. Pubmed Central PMCID: 2743513.
6. Gotink K J, Broxterman H J, Labots M, de Haas R R, Dekker H, Honeywell R J, et al. Lysosomal sequestration of sunitinib: a novel mechanism of drug resistance. Clin Cancer Res. 2011 Dec. 1;17(23):7337-46. PubMed PMID: 21980135.
7. Sawyers C L. Cancer: mixing cocktails. Nature. 2007 Oct. 25;449(7165):993-6. PubMed PM I D: 17960228.
8. Lehar J, Krueger A S, Avery W, Heilbut A M, Johansen L M, Price E R, et al. Synergistic drug combinations tend to improve therapeutically relevant selectivity. Nature biotechnology. 2009 Jul.;27(7):659-66. PubMed PMID: 19581876. Pubmed Central PMCID: 2708317.
9. Bozic I, Reiter JG, Allen B, Antal T, Chatterjee K, Shah P, et al. Evolutionary dynamics of cancer in response to targeted combination therapy. eLife. 2013;2:e00747. PubMed PMID: 23805382. Pubmed Central PMCID: 3691570.
10. Pritchard J R, Lauffenburger D A, Hemann M T. Understanding resistance to combination chemotherapy. Drug resistance updates : reviews and commentaries in antimicrobial and anticancer chemotherapy. 2012 Oct.;15(5-6):249-57. PubMed PMID: 23164555.
11. Kitano H. Towards a theory of biological robustness. Molecular systems biology. 2007;3:137. PubMed PMID: 17882156. Pubmed Central PMCID: 2013924.
12. Messer J, Drabick J, Kaag M. Rational therapy for renal cell carcinoma based on its genetic targets. Adv Exp Med Biol. 2013;779:291-308. PubMed PMID: 23288645. Epub Jan. 5, 2013 eng.
13. Tsutsui H, Valamehr B, Hindoyan A, Qiao R, Ding X, Guo S, et al. An optimized small molecule inhibitor cocktail supports long-term maintenance of human embryonic stem cells. Nat Commun. 2011 Jan;2:167. PubMed PMID: 21266967. Epub Jan. 27, 2011 eng.
14. Chou T C, Martin N. CompuSyn for Drug Combinations: PC Software and User's Guide: A Computer Program for Quantitation of Synergism and Antagonism in Drug Combinations, and the Determination of IC50 and ED50 and LD50 Values. NJ: Paramus; 2005.
15. Nowak-Sliwinska P, Segura T, Iruela-Arispe M L. The chicken chorioallantoic membrane model in biology, medicine and bioengineering. Angiogenesis. 2014;17(4):779-804.
16. Hein M, Graver S. Tumor cell response to bevacizumab single agent therapy in vitro. Cancer cell international. 2013;13(1):94. PubMed PMID: 24059699. Pubmed Central PMCID: 3849065.
17. Bhargava P, Robinson M O. Development of second-generation VEGFR tyrosine kinase inhibitors: current status. Current oncology reports. 2011 Apr.;13(2):103-11. PubMed PMID: 21318618. Pubmed Central PMCID: 3047052. Epub Feb. 15, 2011 eng.
18. Nowak-Sliwinska P, van Beijnum J R, Casini A, Nazarov A A, Wagnieres G, van den Bergh H, et al. Organometallic Ruthenium(II) Arene Compounds with Antiangiogenic Activity. Journal of Medicinal Chemistry. 2011;54(11):3895-902.
19. Adhireksan Z, Davey G E, Campomanes P, Groessl M, Clavel C M, Yu H, et al. A ligand distinctionbetween two ruthenium-cymen anticancer agents determines protein versus DNA targeting. Nature Communications. 2014;5:3462.
20. Bianco R, Garofalo S, Rosa R, Damiano V, Gelardi T, Daniele G, et al. Inhibition of mTOR pathway by everolimus cooperates with EGFR inhibitors in human tumours sensitive and resistant to anti-EGFR drugs. Br J Cancer. 2008 Mar. 11;98(5):923-30. PubMed PMID: 18319715. Pubmed Central PMCID: 2266842.
21. Rao R D, Mladek A C, Lamont J D, Goble J M, Erlichman C, James C D, et al. Disruption of parallel and converging signaling pathways contributes to the synergistic antitumor effects of simultaneous mTOR and EGFR inhibition in GBM cells. Neoplasia. 2005 Oct.;7(10):921-9. PubMed PMID: 16242075. Pubmed Central PMCID: 1502028.
22. Jimeno A, Kulesza P, Wheelhouse J, Chan A, Zhang X, Kincaid E, et al. Dual EGFR and mTOR targeting in squamous cell carcinoma models, and development of early markers of efficacy. Br J Cancer. 2007 Mar. 26;96(6):952-9. PubMed PMID: 17342092. Pubmed Central PMCID: 2360107.
23. Buck E, Eyzaguirre A, Brown E, Petti F, McCormack S, Haley J D, et al. Rapamycin synergizes with the epidermal growth factor receptor inhibitor erlotinib in non-small-cell lung, pancreatic, colon, and breast tumors. Molecular cancer therapeutics. 2006 Nov.;5(11):2676-84. PubMed PMID: 17121914.
24. Wong P K, Yu F, Shahangian A, Cheng G, Sun R, Ho C M. Closed-loop control of cellular functions using combinatory drugs guided by a stochastic search algorithm. Proceedings of the National Academy of Sciences of the United States of America. 2008 Apr 1;105(13):5105-10. PubMed PMID: 18356295. Pubmed Central PMCID: 2278193. Epub Mar. 22, 2008 eng.
25. Jaynes J, Ding X, Xu H, Wong W K, Ho C M. Application of fractional factorial designs to study drug combinations. Statistics in medicine. 2013 Jan. 30;32(2):307-18. PubMed PMID: 22859316.
26. Hinow P, Gerlee P, McCawley L J, Quaranta V, Ciobanu M, Wang S, et al. A spatial model of tumor-host interaction: application of chemotherapy. Mathematical biosciences and engineering : MBE. 2009 Jul.;6(3):521-46. PubMed PMID: 19566124. Pubmed Central PMCID: 2981353.
27. Scolaro C, Bergamo A, Brescacin L, Delfino R, Cocchietto M, Laurenczy G, et al. In vitro and in vivo evaluation of ruthenium(II)-arene PTA complexes. J Med Chem. 2005 Jun. 16;48(12):4161-71. PubMed PMID: 15943488. Epub 2005/06/10. eng.
28. Allardyce CS, Dyson PJ, Ellis DJ, Heath SL. [Ru(eta(6)-p-cymene)CI-2(pta)] (pta=1,3,5-triaza-7-phosphatricyclo[3.3.1.1]decane): a water soluble compound that exhibits pH dependent DNA binding providing selectivity for diseased cells. Chem Commun. 2001 (15):1396-7. PubMed PMID: WOS:000170014800035. English.
29. van Beijnum J R, Dings R P, van der Linden E, Zwaans B M, Ramaekers F C, Mayo K H, et al. Gene expression of tumor angiogenesis dissected: specific targeting of colon cancer angiogenic vasculature. Blood. 2006 Oct. 1;108(7):2339-48. PubMed PMID: 16794251. Epub Jun. 24, 2006 eng.
30. Boyum A. Isolation of lymphocytes, granulocytes and macrophages. Scandinavian journal of immunology. 1976 Jun;Suppl 5:9-15. PubMed PMID: 1052391.
31. van Beijnum J R, Nowak-Sliwinska P, van den Boezem E, Hautvast P, Buurman W A, Griffioen A W. Tumor angiogenesis is enforced by autocrine regulation of high-mobility group box 1. Oncogene. 2013;17(32):363-74.
32. Nazarov A A, Baquie M, Nowak-Sliwinska P, Zava O, van Beijnum J R, Groessl M, et al. Synthesis and characterization of a new class of anti-angiogenic agents based on ruthenium clusters. Scientific Reports. 2013;3:1485, doi: 10.038/srep01485.
33. Yu F, Al-Shyoukh I, Feng J, Li X, Liao C W, Ho C M, et al. Control of Kaposi's sarcoma-associated herpesvirus reactivation induced by multiple signals. PLoS One. 2011;6(6):e20998. PubMed PMID: 21904595. Pubmed Central PMCID: 3125160. Epub Sep. 10, 2011 eng.
34. Sun CP, Usui T, Yu F, Al-Shyoukh I, Shamma J, Sun R, et al. Integrative systems control approach for reactivating Kaposi's sarcoma-associated herpesvirus (KSHV) with combinatory drugs. Integr Biol (Camb). 2009 Jan;1(1):123-30. PubMed PMID: 19851479. Pubmed Central PMCID: 2765122. Epub Oct. 24, 2009 eng.
35. Price K V, Storn R M, Lampinen J A. Differential evolution: a practical approach to global optimization. Berlin: Springer; 2005. xix, 538 p. p.
36. Weiss A, van Beijnum J R, Bonvin D, Jichlinski P, Dyson P J, Griffioen A W, et al. Low-dose angiostatic tyrosine kinase inhibitors improve photodynamic therapy for cancer: lack of vascular normalization. J Cell Mol Med. Jan 22, 2014 PubMed PMID: 24450440.
37. Lim S H, Nowak-Sliwinska P, Kamarulzaman F A, van den Bergh H, Wagnieres G, Lee HB. The neovessel occlusion efficacy of 15-hydroxypurpurin-7-lactone dimethyl ester induced with photodynamic therapy. Photochemistry and photobiology. 2010 Mar.-Apr.;86(2):397-402. PubMed PMID: 20074086. Epub Jan. 16, 2010 eng.
38. Kilarski W W, Muchowicz A, Wachowska M, Mezyk-Kopec R, Golab J, Swartz M A, et al. Optimization and regeneration kinetics of lymphatic-specific photodynamic therapy in the mouse dermis. Angiogenesis. Jul. 28, 2013; Epub ahead of print, PMID:23892627. PubMed PMID: 23892627.
39. Schindelin J, Arganda-Carreras I, Frise E, Kaynig V, Longair M, Pietzsch T, et al. Fiji: an open-source platform for biological-image analysis. Nature methods. 2012 Jul.;9(7):676-82. PubMed PMID: 22743772. Pubmed Central PMCID: 3855844.
40. Gassmann M, Grenacher B, Rohde B, Vogel J. Quantifying Western blots: pitfalls of densitometry. Electrophoresis. 2009 Jun.;30(11):1845-55. PubMed PMID: 19517440.

1-15. (canceled)

16. An angiogenesis inhibitory drug combination obtained by a specific algorithm in which an initial combination of drugs is iteratively adjusted.

17. The drug combination according to claim 16 obtained according to an FSC technique.

18. The drug combination according to claim 16, further comprising a RAPTA-C compound.

19. The drug combination according to claim 18 for the treatment of ovarian carcinoma.

20. The drug combination according to claim 18 for the treatment of colorectal adenocarcinoma.

21. The drug combination according to claim 18 for the treatment of other neoplasms or other proliferative diseases.

22. The drug combination according to claim 18, further comprising erlotinib.

23. The drug combination according to claim 22, further comprising BEZ235.

24. The drug combination according to claim 16, further comprising:
a ruthenium-arene based compound of general formula Ru(arene)(X)(Y)(Z)⁺, where R=any organic group and X, Y and Z=any ligand including chelating ligands and +=0, 1 or 2.

25. The drug combination according to claim 16 for the suppression of microvessel density in tumors.

26. The drug combination according to claim 16 for inducing cell stasis, cell death or apoptosis in activated (tumor-) endothelial cells.

27. A method of identifying a drug dosage combination comprising the steps of:
iteratively adjusting a combination of drugs; and
identifying an optimal angiogenesis inhibitory or cytostatic drug combination with decreased adverse side effects.

28. A method of identifying a drug dosage combination comprising the steps of:
iteratively adjusting a combination of drugs; and
identifying an optimal angiogenesis inhibitory or cytostatic drug combination with decreased drug-induced resistance.

29. A method of searching for an optimized combination of drugs comprising the step of:
using endothelial cell proliferation as a read out to navigate in a parametric space to screen for combinations of the drugs.

30. A method of searching for an optimized combination of drugs comprising the step of:
using an algorithm to identify an optimal angiogenesis inhibitory or cytostatic drug combination with from a parametric space.
